(19) United States
(12) Patent Application Publication
(10) Pub. No.: US 2025/0290161 A1
(43) Pub. Date: Sep. 18, 2025

(54) DCAPS MOLECULAR MARKER FOR IDENTIFYING RESISTANCE OF WHEAT TO FUSARIUM CROWN ROT CAUSED BY FUSARIUM PSEUDOGRAMINEARUM, PRIMERS AND APPLICATION THEREOF

(71) Applicant: Henan Agricultural University, Zhengzhou (CN)

(72) Inventors: Qiaoyun Li, Zhengzhou (CN); Guihong Yin, Zhengzhou (CN); Xiaopeng Hao, Zhengzhou (CN); Zhenfeng Guo, Zhengzhou (CN); Kefei Qu, Zhengzhou (CN); Jianwei Tang, Zhengzhou (CN); Chunhao Dong, Zhengzhou (CN); Yuhao Yuan, Zhengzhou (CN); Zhenpu Huang, Zhengzhou (CN); Yan Gao, Zhengzhou (CN)

(21) Appl. No.: 18/632,319

(22) Filed: Apr. 11, 2024

(30) Foreign Application Priority Data
Mar. 13, 2024 (CN) 202410285234.9

(51) Int. Cl. C12Q 1/6895

(52) U.S. Cl. CPC C12Q 1/6895; C12Q 2600/13; C12Q 2600/156

(57) ABSTRACT

A derived cleaved amplified polymorphic sequence (dCAPS) molecular marker for identifying resistance of wheat to Fusarium crown rot caused by Fusarium pseudograminearum, primers and an application thereof are provided, relating to the field of wheat disease-resistant breeding and biotechnologies. The dCAPS molecular marker is located at a site of 676077050 to 676084414 base pairs (bp) on wheat chromosome 1B. A primer set for identifying the dCAPS molecular marker is provided. The resistance to Fusarium crown rot caused by Fusarium pseudograminearum can be rapidly predicted and screened through the molecular marker detection during seedling stage, which saves precious scientific research time and a lot of manpower and material resources, and the identification result is stable. Therefore, the method can accurately and efficiently screen a wheat variety with resistance to the Fusarium crown rot, and greatly improve a breeding process of a wheat with high-yield and resistance to the Fusarium crown rot.

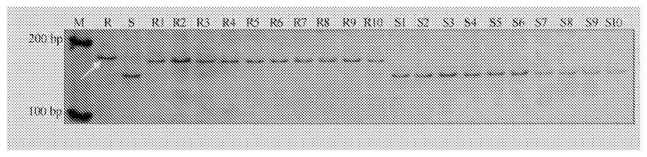

### TECHNICAL FIELD

The disclosure relates to the field of wheat disease-resistant breeding and biotechnologies, and more particularly to a derived cleaved amplified polymorphic sequence (dCAPS) molecular marker for identifying resistance of wheat to Fusarium crown rot (FCR) caused by Fusarium pseudograminearum, primers and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 24020TBYX-USP1-SL.xml. The XML file is 2,912 bytes; is created on Mar. 28, 2024; and is being submitted electronically via patent center.

### BACKGROUND

Fusarium crown rot (FCR) of wheat is a soil-borne disease caused by Fusarium fungi, which can occur from a seedling stage to an adult plant stage (also referred to as mature-plant stage) of wheat, causing browning of seedling base and leaf sheath of the wheat and even death of an entire plant of the wheat, browning and necrosis of stem of the wheat after jointing, and withering and white spikes in severe cases. The FCR of wheat causes 20% to 50% reduction in production, and multiple mycotoxins such as Deoxynivalenol (DON) and Zearalenone (ZEN) are remained in plants and seeds infected by the FCR, which pose a threat to human and animal life and health. Pathogens of the FCR include multiple Fusarium fungi such as Fusarium pseudograminearum, Fusarium graminearum (F. graminearum), and Fusarium culmorum (F. culmorum), and the Fusarium pseudograminearum is a dominant pathogen in Huang-Huai wheat area of China.

The FCR of wheat becomes a globally important disease since it was reported in Australia in the 1950s. In recent years, the FCR shows a trend of continuous spread and aggravation in China. For example, in Henan province, white spike rates of diseased fields caused by the FCR in 2013 and 2016 are respectively in a range of 0.4% to 11.0% and a range of 0.1% to 31.5%. In 2019, an incidence rate of some fields in Henan province is as high as 78.9%, and yield loss of some diseased fields is as high as 51.6%. In 2021, the FCR is serious in many places in Henan province, which reduces a yield by 20% to 30%, and reduces the yield by more than 50% in serious areas. According to statistics of National Agricultural Technology Extension Service Center, an incidence area of the FCR in China is 23.45 million mu (fifteen mu is equal to one hectare) in 2020, 30.78 million mu in 2021, and over 40 million mu annually in 2022 and 2023. Specifically, main wheat-producing areas in Huang-Huai and North China are aggravating. It can be seen that the FCR becomes an important problem to be solved urgently in wheat production in China.

The pathogens of the FCR have a wide range of hosts and can survive for several years on residues such as corn and wheat in soil. In addition, the FCR of wheat has characteristics of occurring at a base of the stem and can occur throughout an entire growth period, making prevention and control of this disease long-term and difficult. At present, main measures to deal with the FCR of wheat in production are chemical drug prevention and treatment, which has unsatisfactory prevention and treatment effects, and brings environmental pollution problems, at the same time increases production costs, and burning residues, crop rotation and other measures cannot fully control the FCR. Planting a disease-resistant variety is the most economical and effective measure to deal with the FCR. However, at present, there is a lack of idioplasm that is immune and highly resistant to the FCR, and there are also very few idioplasm that have moderate resistance to the FCR. More than 90% of wheat varieties in production are susceptible to the FCR, so it is necessary to increase breeding of a new wheat variety with resistance to the FCR.

The breeding of the disease-resistant variety needs a clear genetic law of resistance to guide a configuration of hybrid combinations and accurate phenotypic identification for screening of offspring. Researchers at home and abroad have done a lot of research on a genetic law of resistance to the FCR, and found that the resistance of wheat to the FCR is a quantitative trait, and more than 100 quantitative trait loci (QTL)/genes are located on 21 chromosomes of the wheat at present. Major QTL or aggregated minor QTL can be transformed in a genome of a high-yielding susceptible wheat variety through molecular marker-assisted selection, thus improving their resistance to the FCR, and accelerating a process of the breeding of the new wheat variety resistant to the FCR. However, there is a lack of a practical molecular marker for the resistance to the FCR in the wheat breeding at present.

In addition, a standard for identifying resistance to the FCR of wheat cannot be unified, and inoculation periods, inoculation methods and evaluation indexes are quite different. For example, the inoculation periods include the seedling stage and the adult stage, and there are two seedling cultivation methods during the seedling stage, including: soil cultivation and hydroponics. The inoculation methods mainly include a stem base instillation method, a natural culture medium method and a spore liquid immersion method. The evaluation indexes are different, including a symptom grading method that record a degree of browning, a severity index that consider a number of layers of leaf sheath browning, a leaf sheath symptom score, and the like. Evaluation criteria of resistance are also different. For example, when a disease index based on the symptom grading method is 35, some of them are evaluated as a highly susceptible idioplasm and some are evaluated as a moderately resistant idioplasm. In addition, occurrence and development of the FCR are affected by the environment, which leads to poor stability of the identification results of the same wheat variety.

### SUMMARY

In order to solve the above problems, the disclosure provides a derived cleaved amplified polymorphic sequence (dCAPS) molecular marker for identifying resistance of wheat to Fusarium crown rot (FCR) caused by Fusarium pseudograminearum, and the dCAPS molecular marker is located at a site of 676077050 to 676084414 base pairs (bp) on a wheat chromosome 1B.

The disclosure further provides a primer set, and the primer set is configured to amplify the above dCAPS molecular marker.

In an embodiment, the primer set includes: an upstream primer and a downstream primer; and the nucleotide sequence of the upstream primer is shown as SEQ ID NO: 1, and the nucleotide sequence of the downstream primer is shown as SEQ ID NO: 2.

The disclosure further provides a detection reagent for identifying the above dCAPS molecular marker, and the detection reagent includes the above primer set.

The disclosure further provides a detection kit for identifying the above dCAPS molecular marker, and the detection kit includes the above primer set or the above detection reagent.

The disclosure further provides an application method of the above dCAPS molecular marker, and the application method includes:
identifying the resistance of wheat to the Fusarium crown rot caused by the Fusarium pseudograminearum by using the above dCAPS molecular marker.

The disclosure further provides a method for identifying the resistance of wheat to the Fusarium crown rot caused by the Fusarium pseudograminearum, and the method includes:
taking a genomic DNA of a wheat sample as a template, performing a polymerase chain reaction (PCR) amplification with the above primer set to obtain a PCR amplification product, digesting the PCR amplification product to obtain a digested product, and performing an electrophoresis on the digested product to obtain an electrophoresis product; when the electrophoresis product includes a DNA fragment with 169 bp, indicating that the wheat sample is a resistant variety to the Fusarium crown rot caused by the Fusarium pseudograminearum.

Steps of the digesting includes:
mixing 0.5 microliters (μL) of 10 units per microliter abbreviated as U/μL of PvuII enzyme ( ), 1.0 μL of 10×M buffer, 1.0 μL of the PCR amplification product and 7.5 μL of double-distilled water (ddH₂O) to obtain a mixture, digesting the mixture at a temperature of 37.0 Celsius degree (° C.) for 4 hours (h) to obtain a digested mixture, and adding 1.0 μL of loading buffer into the digested mixture to obtain the digested product.

Steps of the electrophoresis includes:
performing a polyacrylamide gel electrophoresis with a mass fraction of 8% on 2 μL of the digested product to obtain the electrophoresis product;
when the electrophoresis product includes the DNA fragment with 169 bp, indicating that the wheat sample has a target dCAPS molecular marker, and predicting that the wheat sample has resistance to the Fusarium crown rot;
when the electrophoresis product does not include the DNA fragment with 169 bp, indicating that the wheat sample does not have the resistance to the Fusarium crown rot.

In an embodiment, the 10×M buffer includes: 100 millimoles per liter (mM) of tris(hydroxymethyl)aminomethane hydrochloride (Tris-HCl) with pH of 7.5, 100 mM of magnesium chloride (MgCl₂), 10 mM of dithiothreitol, and 500 mM of sodium chloride (NaCl).

Beneficial effects of the disclosure are as follows.

Compared to a conventional field identification method, in the disclosure, the resistance of wheat to the Fusarium crown rot caused by the Fusarium pseudograminearum are predicted and screened through a molecular marker detection during a seedling stage of the wheat, and innovation and beneficial effects of the disclosure are specifically reflected in the following three aspects.

In a first aspect, an identification time is saved. 30 to 100 days (d) need to be used to detect the resistance of wheat to the Fusarium crown rot according to different methods, while the method of the disclosure utilizes the molecular marker to perform a genotype detection, which is not limited by seasons and can be performed from a seedling stage to an adult plant stage of the wheat, and only 2 d are taken from sampling to DNA extraction to the polyacrylamide gel electrophoresis for color development, thus, the method of the disclosure greatly shortens the identification time. The identification result of the disclosure can guide a preparation of hybrid combinations for the current year, so that hybridization can be carried out one year in advance. Therefore, the most remarkable innovation and beneficial effect of the method of the disclosure are that it is not limited by seasons and can be quickly identified.

In a second aspect, the identification result is stable. In the method of the disclosure, the resistance of wheat to the Fusarium crown rot is predicted and screened through detecting the molecular marker, which overcomes a phenomenon that the identification result is unstable since the resistance of wheat to the Fusarium crown rot is easily affected by environment in conventional breeding, and the PCR amplification method is stable, simple to operate and convenient for different units to use.

In a third aspect, manpower and material resources are saved. Compared to complicated links such as pathogen preservation and propagation, preparation of diseased grains or spore liquid, pathogen inoculation, plant culture and disease condition investigation of a conventional identification for the resistance to the Fusarium crown rot, the method of the disclosure only needs the DNA extraction, the PCR amplification, the digesting and the polyacrylamide gel electrophoresis, thus saving a large amount of manpower and material resources.

In method of the disclosure, the resistance of wheat to the Fusarium crown rot caused by the Fusarium pseudograminearum can be rapidly predicted and screened through the molecular marker detection during the seedling stage of the wheat, which saves precious scientific research time and a lot of manpower and material resources, and the identification result is stable. Therefore, the method of the disclosure can accurately and efficiently screen a wheat variety with resistance to the Fusarium crown rot, so as to greatly improve a breeding process of a wheat with high-yield and resistance to the Fusarium crown rot.

### BRIEF DESCRIPTION OF DRAWING

In order to provide a clearer description of technical solutions in embodiments of the disclosure or related art, drawing required in the embodiments will be simply introduced below. Apparently, the drawing in the following descriptions are merely one of the embodiments, for those skilled in the art, other drawings can be obtained according to the drawing without creative work.

FIGURE illustrates a schematic diagram of a detection result of an FCR-1B-d8 molecular marker on a F₂ population by using a method of the disclosure.

In the figure, M represents a molecular weight standard (100 bp DNA ladder), R represents a disease-resistant line fcrZ22, S represents a susceptible variety Zhoumai 22, R1 to R10 represent different resistant strains of the F₂ population, and S1 to S10 represent different susceptible strains; a white arrow indicates the FCR-1B-d8 molecular marker amplified from the disease-resistant line fcrZ22, a size of the FCR-1B-d8 molecular marker is 169 bp, and the FCR-1B-d8 molecular marker is closely linked to resistance QTL [symbol] CR.hau-1B to Fusarium crown rot of wheat caused by Fusarium pseudograminearum; a corresponding position of the Zhoumai 22 does not have the FCR-1B-d8 molecular marker, and the 10 resistant strains R1 to R10 carry the FCR-1B-d8 molecular marker, while the 10 susceptible strains S1 to S10 do not carry the FCR-1B-d8 molecular marker.

### DETAILED DESCRIPTION OF EMBODIMENTS

Various embodiments of the disclosure are described in detail. Unless otherwise specified, methods in the embodiments are conventional methods, and unless otherwise specified, reagents used are conventional commercially available reagents or reagents prepared by conventional methods. This detailed description should not be construed as a limitation of the disclosure but rather as a more detailed description of certain aspects, features and embodiments of the disclosure.

It should be understood that terms described in the disclosure are only for describing specific embodiments and are not intended to limit the disclosure. Furthermore, for a numerical range in the disclosure, it should be understood that each intermediate value between upper and lower limits of the numerical range is also specifically disclosed. Each smaller range between any stated value or intermediate value within a stated range, as well as any other stated value or intermediate value within the stated range, is also included in the disclosure. The upper and lower limits of these smaller ranges can be independently included or excluded from the stated range.

Unless otherwise specified, all technical and scientific terms used in this article have the same meanings as those commonly understood by those skilled in the art described herein. Although the disclosure only describes some methods and materials, any methods and materials similar or equivalent to those described herein may also be used in the embodiments or testing of the disclosure. All literature mentioned in the specification is incorporated by reference to publicly disclose and describe methods and/or materials related to the literature. In case of conflict with any incorporated literature, a content of the specification shall prevail.

It is obvious to those skilled in the art that many improvements and changes can be made to the specific embodiments of the disclosure without departing from a scope or spirit of the disclosure. Other embodiments will be apparent to those skilled in the art from the descriptions of the disclosure. The descriptions and embodiments of the disclosure are exemplary only.

Terms “including”, “comprising”, “having” and “containing” used in this article are all open terms, which means including but not limited to.

Explanations of the disclosure are as follows.
(1) Sequences of a primer pair are as follows:

```
  (as shown in SEQ ID NO: 1)
FCR-1B-8F: 5′-TTTGCTTAGTTTATTAAAAGCAGCT-3′;
and
  (as shown in SEQ ID NO: 2)
FCR-1B-8R: 5′-CACTCTGTTCCCATCATCAC-3′.
```

(2) A preparation of an 8% polyacrylamide gel solution: 1 liter (L) of the polyacrylamide gel solution includes 267 milliliters (mL) of 30% acrylamide/bisacrylamide (Acry/Bis) solution, 200 mL of 5×tris-borate-ethylene diamine tetraacetic acid abbreviated as EDTA (TBE) buffer and 534 mL of double-distilled water (ddH₂O).

### Embodiment 1

A disease-resistant line fcrZ22 is hybridized with a wheat susceptible variety Zhoumai 22, and the obtained seeds are selfed to obtain F₂ plants. An inoculation identification is performed on a segregating F₂ population to select 20 extremely resistant strains and 20 extremely susceptible strains, to thereby construct a resistant pool and a susceptible pool. Combined with the genotype analysis of resistant parents, susceptible parents, the resistant pool and the susceptible pool through a 660K chip, single nucleotide polymorphisms (SNPs) associated with the resistance to the Fusarium crown rot are detected. The most associated SNPs are detected on the chromosome 1B. The SNP marker on the chromosome 1B is converted to a dCAPS marker to perform the molecular detection on the F₂ plants. A QTL IciMapping V4.1 drawing software is used to construct a genetic linkage map, and the inoculation identification is simultaneously preformed on F_{2:3} strains to obtain identification data for resistance of the F_{2:3} strains to the Fusarium crown rot. The genetic linkage map constructed by the molecular detection on the F₂ plants and the identification data for resistance of the F_{2:3} strains to the Fusarium crown rot are combined to position a resistant QTL [symbol] CR.hau-1B to the Fusarium crown rot on the chromosome 1B and obtain a molecular marker closely linked to the resistant QTL [symbol] CR.hau-1B. An FCR-1B-d8 marker shown in the disclosure is the molecular marker closely linked to the resistant QTL [symbol] CR.hau-1B on the chromosome 1B of the fcrZ22. In 2021, the molecular detection of the FCR-1B-d8 marker was performed on typical resistant and susceptible strains in the F₂ population. A specific detection method are as follows.
a. Sample collection: during a three-leaf stage of each sample, leaves of the fcrZ22, the Zhoumai 22, 10 resistant strains and 10 susceptible strains with lengths of 1.5 centimeters (cm) are taken, the leaves are packed into 1.5 mL sterilized centrifuge tubes respectively, and the sterilized centrifuge tubes with the leaves are placed into an ice box and brought back to a laboratory for extracting DNA.
b. DNA extraction: DNA of the samples are extracted by using a hexadecyl trimethyl ammonium bromide (CTAB) method (Wang Guanlin, Fang Hongjun, Plant genetic engineering principle and technology, Science Press, 1998, pages 370-372) to thereby obtain dried DNA precipitate, the dried DNA precipitate is dissolved in 50 microliters (μL) of tris-EDTA (TE) buffer solution to obtain wheat sample DNA, and then the wheat sample DNA is stored at a temperature of −20° C. for later use.
c. PCR amplification: a PCR reaction system includes 10 μL total reaction volume, specifically including: 5 μL of 2×Tag Master Mix (No Dye), 0.5 μL of FCR-1B-8F primer with a concentration of 10 micromoles per liter (μmol/L), 0.5 μL of FCR-1B-8R primer with a concentration of 10 μmol/L, 1.0 μL of the wheat sample DNA with a concentration of 50 nanograms per microliter (ng/μL), and 3 μL of ddH₂O.

PCR amplification procedures include: pre-denaturation at a temperature of 95° C. for 3 minutes (min); a total of 35 cycles of denaturation at a temperature of 95° C. for 15 seconds(s), annealing at a temperature of 59° C. for 15 s and extension at a temperature of 72° C. for 30 s; and final extension at a temperature 72° C. for 5 min, thus, an amplification product is obtained.
d. 1.0 μL of the amplification product is digested: a restriction endonuclease is PvuII enzyme, a digesting system includes 10 μL total reaction volume, and specifically including: 0.5 μL of the Pvull enzyme (10 U/μL), 1.0 μL of 10×M buffer, 1.0 μL of the PCR amplification product (i.e., the amplification product of the step c) and 7.5 μL of ddH₂O. The amplification product is digested at a temperature of 37.0° C. for 4 h to obtain a digested product.
e. Electrophoresis and result observation: 1.0 μL of 10×loading buffer is added into the digested product and stirred evenly, and an 8% polyacrylamide gel electrophoresis is performed on 2 μL of the digested product added with the 10×loading buffer at 500 volts (V) for 1 h to obtain an electrophoresis product, and the electrophoresis product is stained by a silver staining method.
f. A result of the electrophoresis is shown in the figure, M represents a molecular weight standard (100 bp DNA ladder), R represents the disease-resistant line fcrZ22, S represents a susceptible variety Zhoumai 22, R1 to R10 represent different resistant strains of the F₂ population, and S1 to S10 represent different susceptible strains of the F₂ population. A white arrow in the figure indicates the FCR-1B-d8 molecular marker amplified from the disease-resistant line fcrZ22, a size of the FCR-1B-d8 molecular marker is 169 bp, and the FCR-1B-d8 molecular marker is closely linked to the resistance QTL [symbol] CR.hau-1B to the Fusarium crown rot of wheat caused by Fusarium pseudograminearum. A corresponding position of the Zhoumai 22 does not have the FCR-1B-d8 molecular marker, and the 10 resistant strains R1 to R10 carry the FCR-1B-d8 molecular marker, while the 10 susceptible strains S1 to S10 do not carry the FCR-1B-d8 molecular marker.

### Embodiment 2

In March 2022, resistance of a F₆ population for the Fusarium crown rot caused by the Fusarium pseudograminearum is rapidly predicted by using the method of the disclosure. A female parent of the F₆ population is a high-yield variety Yunong 903, a male parent of the F₆ population is the resistant line fcrZ22, 130 strains of the F₆ population are customized as YN903-FCR-1 to YN903-FCR-130, and specific detection steps are as follows.
a. Sample collection: healthy seeds of 130 strains of the F₆ population are taken (2 seeds per strain), crushed by using a hammer head, and then packed into the 1.5 mL sterilized centrifuge tube for extracting DNA.
b. DNA extraction: DNA in the samples in the step a are extracted by using a CTAB method (Wang Guanlin, Fang Hongjun, Plant genetic engineering principle and technology, Science Press, 1998, pages 370-372) to thereby obtain dried DNA precipitate. The dried DNA precipitate is dissolved in 50 μL of the TE buffer solution to obtain wheat sample DNA, and the wheat sample DNA is stored at a temperature of −20° C. for later use.
c. PCR amplification: a PCR reaction system includes 10 μL total reaction volume, specifically including: 5 μL of 2×Tag Master Mix (No Dye), 0.5 μL of FCR-1B-8F primer with a concentration of 10 μmol/L, 0.5 μL of FCR-1B-8R primer with a concentration of 10 μmol/L, 1.0 μL of the wheat sample DNA with a concentration of 50 ng/μL and 3 μL of ddH₂O.

A PCR amplification procedure includes: pre-denaturation at a temperature of 95° C. for 3 min; a total of 35 cycles of denaturation at a temperature of 95° C. for 15 s, annealing at a temperature of 59° C. for 15 s and extension at a temperature of 72° C. for 30 s; and final extension at a temperature 72°° C. for 5 min, thus, an amplification product is obtained.
d. 1.0 μL of the amplification product is digested. A restriction endonuclease is Pvull enzyme, a digesting system includes 10 μL total reaction volume, and specifically including: 0.5 μL of Pvull enzyme (10 U/μL), 1.0 μL of 10×M buffer, 1.0 μL of the PCR amplification product (i.e., the amplification product of the step c) and 7.5 μL of ddH₂O. The amplification product is digested at a temperature of 37.0° C. for 4 h to obtain a digested product.
e. Electrophoresis and result observation: 1.0 μL of 10×loading buffer is added into the digested product and stirred evenly, and an 8% polyacrylamide gel electrophoresis is performed on 2 μL of the digested product added with the 10×loading buffer at 500 V for 1 h to obtain an electrophoresis product, and the electrophoresis product is stained by the silver staining method. The amplification product is separated by performing the 8% polyacrylamide gel electrophoresis to obtain the electrophoresis product, and the electrophoresis product is inspected whether it carries the FCR-1B-d8 molecular marker with 169 bp. When the electrophoresis product carries the FCR-1B-d8 molecular marker, it can be predicted that the wheat seedling has resistance to the Fusarium crown rot (Table 1) according to the results of the F₂ population analysis in the Zhoumai 22/fcrZ22.
f. A natural medium inoculation (diseased grains) method commonly used in identification of the resistance of wheat to the Fusarium crown rot at the seedling stage (Zhou et al., Diversity of the Fusarium pathogens associated with crown rot in the Huanghuai wheat-growing region of China, Environ Microbiol, 2019, pages 2740-2754, volume 21; Yang Yun et al., Resistance of wheat cultivars in Huang-Huai region of China to crown rot caused by Fusarium pseudograminearum, journal of triticeae crops, 2015, pages 339-345, volume 35) is used to identify resistance of the YN903-FCR-1 to YN903-FCR-130 strains to the Fusarium crown rot caused by the Fusarium pseudograminearum, and 10 plants per strain are identified. Incidences of the Fusarium crown rot are investigated according to a 0 to 9 grade method to calculate an average disease index, and a calculation method of the average disease index is as follows: disease index (DI)=Σ[(grades×number of plants of each grade)/(highest grade×total number of plants)]×100.
g. The detection result in the step e using the FCR-1B-d8 molecular marker is compared to the actual identification result of the resistance to the Fusarium crown rot in the step f, and a comparative result is shown in Table 1. It can be seen from Table 1 that 89 of the 130 strains do not carry the FCR-1B-d8 molecular marker, and the average disease index is 46.1%. There are 41 strains carry the FCR-1B-d8 molecular marker, and the average disease index is 24.7%. The disease index of the strains carrying the FCR-1B-d8 molecular marker is significantly lower than that of the strains without the FCR-1B-d8 molecular marker, which indicates that introducing the FCR-1B-d8 molecular marker into genome of high-yield varieties could significantly improve the resistance to the Fusarium crown rot.

TABLE 1
Detection results of the molecular marker in strains of the F₆ population
and identification results of the resistance to the Fusarium crown rot

| | Marker | Disease | | Marker | Disease | | Marker | Disease |
|---|---|---|---|---|---|---|---|---|
| Stain | detection | index | Strain | detection | index | Strain | detection | index |
| YN903-FCR-1 | + | 25.6 | YN903-FCR-5 | − | 41.1 | YN903-FCR-66 | − | 29.4 |
| YN903-FCR-4 | + | 26.9 | YN903-FCR-8 | − | 41.5 | YN903-FCR-70 | − | 49.1 |
| YN903-FCR-6 | + | 27.1 | YN903-FCR-10 | − | 42.2 | YN903-FCR-71 | − | 49.8 |
| YN903-FCR-7 | + | 19.2 | YN903-FCR-11 | − | 42.3 | YN903-FCR-72 | − | 55.8 |
| YN903-FCR-9 | + | 28.2 | YN903-FCR-12 | − | 43.5 | YN903-FCR-73 | − | 45.3 |
| YN903-FCR-16 | + | 24.5 | YN903-FCR-13 | − | 44.2 | YN903-FCR-74 | − | 46.5 |
| YN903-FCR-19 | + | 29.6 | YN903-FCR-14 | − | 44.9 | YN903-FCR-76 | − | 47.2 |
| YN903-FCR-21 | + | 30.6 | YN903-FCR-15 | − | 45.6 | YN903-FCR-78 | − | 47.9 |
| YN903-FCR-31 | + | 25.2 | YN903-FCR-17 | − | 46.1 | YN903-FCR-79 | − | 48.6 |
| YN903-FCR-32 | + | 25.8 | YN903-FCR-18 | − | 46.8 | YN903-FCR-80 | − | 49.1 |
| YN903-FCR-33 | + | 26.6 | YN903-FCR-20 | − | 52.8 | YN903-FCR-87 | − | 49.8 |
| YN903-FCR-43 | + | 19.8 | YN903-FCR-22 | − | 32.1 | YN903-FCR-88 | − | 55.8 |
| YN903-FCR-44 | + | 19.4 | YN903-FCR-23 | − | 43.5 | YN903-FCR-90 | − | 57.8 |
| YN903-FCR-52 | + | 27.6 | YN903-FCR-24 | − | 44.2 | YN903-FCR-91 | − | 47.3 |
| YN903-FCR-53 | + | 28.1 | YN903-FCR-25 | − | 44.9 | YN903-FCR-93 | − | 48.5 |
| YN903-FCR-54 | + | 20.0 | YN903-FCR-26 | − | 45.6 | YN903-FCR-96 | − | 49.2 |
| YN903-FCR-55 | + | 24.6 | YN903-FCR-27 | − | 46.1 | YN903-FCR-97 | − | 49.9 |
| YN903-FCR-59 | + | 18.6 | YN903-FCR-28 | − | 46.8 | YN903-FCR-101 | − | 50.6 |
| YN903-FCR-65 | + | 28.6 | YN903-FCR-29 | − | 52.8 | YN903-FCR-103 | − | 51.1 |
| YN903-FCR-67 | + | 25.9 | YN903-FCR-30 | − | 42.3 | YN903-FCR-104 | − | 51.8 |
| YN903-FCR-68 | + | 26.1 | YN903-FCR-34 | − | 43.5 | YN903-FCR-105 | − | 57.8 |
| YN903-FCR-69 | + | 29.7 | YN903-FCR-35 | − | 44.2 | YN903-FCR-109 | − | 35.8 |
| YN903-FCR-75 | + | 19.1 | YN903-FCR-36 | − | 44.9 | YN903-FCR-110 | − | 46.2 |
| YN903-FCR-77 | + | 31.2 | YN903-FCR-37 | − | 45.6 | YN903-FCR-111 | − | 40.1 |
| YN903-FCR-81 | + | 18.4 | YN903-FCR-38 | − | 46.1 | YN903-FCR-112 | − | 53.2 |
| YN903-FCR-82 | + | 26.7 | YN903-FCR-39 | − | 46.8 | YN903-FCR-113 | − | 31.4 |
| YN903-FCR-83 | + | 27.2 | YN903-FCR-40 | − | 52.8 | YN903-FCR-114 | − | 42.5 |
| YN903-FCR-84 | + | 27.8 | YN903-FCR-41 | − | 41.7 | YN903-FCR-115 | − | 37.8 |
| YN903-FCR-85 | + | 28.6 | YN903-FCR-42 | − | 42.6 | YN903-FCR-116 | − | 52.9 |
| YN903-FCR-86 | + | 20.1 | YN903-FCR-45 | − | 43.1 | YN903-FCR-117 | − | 61.7 |
| YN903-FCR-89 | + | 18.7 | YN903-FCR-46 | − | 43.5 | YN903-FCR-118 | − | 40.4 |
| YN903-FCR-92 | + | 23.6 | YN903-FCR-47 | − | 44.2 | YN903-FCR-119 | − | 41.0 |
| YN903-FCR-94 | + | 29.6 | YN903-FCR-48 | − | 44.3 | YN903-FCR-120 | − | 46.8 |
| YN903-FCR-95 | + | 21.7 | YN903-FCR-49 | − | 45.5 | YN903-FCR-121 | − | 29.8 |
| YN903-FCR-98 | + | 18.1 | YN903-FCR-50 | − | 46.2 | YN903-FCR-122 | − | 30.4 |
| YN903-FCR-99 | + | 24.9 | YN903-FCR-51 | − | 27.5 | YN903-FCR-123 | − | 62.4 |
| YN903-FCR-100 | + | 30.6 | YN903-FCR-56 | − | 47.6 | YN903-FCR-124 | − | 47.6 |
| YN903-FCR-102 | + | 25.1 | YN903-FCR-57 | − | 68.1 | YN903-FCR-125 | − | 51.7 |
| YN903-FCR-106 | + | 25.7 | YN903-FCR-58 | − | 48.8 | YN903-FCR-126 | − | 48.6 |
| YN903-FCR-107 | + | 17.8 | YN903-FCR-60 | − | 54.8 | YN903-FCR-127 | − | 36.5 |
| YN903-FCR-108 | + | 21.4 | YN903-FCR-61 | − | 44.3 | YN903-FCR-128 | − | 39.1 |
| Average | | 24.7 | YN903-FCR-62 | − | 45.5 | YN903-FCR-129 | − | 42.5 |
| YN903-FCR-2 | − | 39.7 | YN903-FCR-63 | − | 47.2 | YN903-FCR-130 | − | 45.3 |
| YN903-FCR-3 | − | 40.6 | YN903-FCR-64 | − | 47.9 | Average | − | 46.1 |

Note:
(1) + represents presence of the FCR-1B-d8 molecular marker; and − represents absence of the FCR-1B-d8 molecular marker.
(2) The resistance identification test is made in an artificial climate room of Henan Agricultural University.
(3) The identification results using the method of the disclosure show that 89 of the 130 strains do not carry the FCR-1B-d8 molecular marker, and the average disease index is 46.1%.

There are 41 strains carry the FCR-1B-d8 molecular marker, and an average black embryo rate (i.e., the average disease index) is 24.7%. which indicates that introducing the FCR-1B-d8 molecular marker into the genome of the high-yield variety Yuning 903 could significantly improve the resistance to the Fusarium crown rot.

### Embodiment 3 High Throughput Rapid Detection to Improve Breeding Efficiency

Detection materials are 500 individual plants in a BC₁ population, and the BC₁ population is obtained by crossing the high-yielding variety Zhoumai 36 (female parent) with the disease resistant line fcrZ22 (male parent) and backcrossing it once again. The 500 BC₁ individual plants are customized as ZZ-FCR-1 to ZZ-FCR-500.

Detection steps are basically the same as the embodiment 1, and differences between the embodiment 3 and the embodiment 1 are as follows.

In step a, the sample collection stage is changed from the three-leaf stage to a jointing stage, during which plants can form panicles and aid in preliminary observation of agronomic traits.

In step b, the dried DNA precipitate is dissolved in 50 μL of ddH₂O and allowed to stand at a room temperature for 30 min, and then stored at a temperature of −20° C. for later use. The detection time is very short, the DNA does not need to be stored for a long time, low cost ddH₂O can be used to dissolve DNA.

In March 2023, a molecular detection was performed on the 500 BC₁ individual plants preliminary screened according to the agronomic traits. Only 7 d are taken from DNA extraction solution preparation, sampling to obtaining test results. Among the 500 individual plants, 109 are detected with the FCR-1B-d8 molecular marker (as shown in Table 2). When a conventional resistance identification method is used, it takes more than 30 d, and diseased grains need to be prepared in advance, sterilized soil needs to be prepared, and a climate room is needed, which leads to many breeding units not being able to conduct the resistance identification for the Fusarium crown rot. It is a common method to obtain a high-yield and disease-resistant wheat by crossing a disease-resistant strain with a high-yield variety, backcrossing and then screening a disease-resistant plant from BC₁ population and continuing backcrossing with the high-yield parent, and phenotypic identification is a premise of screening the disease-resistant plant. The conventional identification method of the resistance to the Fusarium crown rot needs a long time, complicated procedures, and cannot detect a large number of breeding offspring, and general breeding units do not have the detection conditions. The molecular marker-assisted detection selection method of the disclosure can be carried out in large quantities at the same time, and can be carried out in general units, with short time and simple procedures, which is beneficial to improving the breeding efficiency of a new wheat variety with resistance to the Fusarium crown rot.

TABLE 2
Molecular detection results of 500 BC₁ individual plants

| Plant number | Marker detection |
|---|---|
| ZZ- FCR -1 | − |
| ZZ- FCR -2 | + |
| ZZ- FCR -3 | − |
| ZZ- FCR -4 | − |
| ZZ- FCR -5 | + |
| ZZ- FCR -6 | + |
| ZZ- FCR -7 | − |
| ZZ- FCR -8 | − |
| ZZ- FCR -9 | − |
| ZZ- FCR -10 | + |
| ZZ- FCR -11 | − |
| ZZ- FCR -12 | − |
| ZZ- FCR -13 | − |
| ZZ- FCR -14 | − |
| ZZ- FCR -15 | + |
| ZZ- FCR -16 | − |
| ZZ- FCR -17 | − |
| ZZ- FCR -18 | − |
| ZZ- FCR -19 | + |
| ZZ- FCR -20 | + |
| ZZ- FCR -21 | − |
| ZZ- FCR -22 | − |
| ZZ- FCR -23 | − |
| ZZ- FCR -24 | + |
| ZZ- FCR -25 | + |
| ZZ- FCR -26 | − |
| ZZ- FCR -27 | − |
| ZZ- FCR -28 | − |
| ZZ- FCR -29 | − |
| ZZ- FCR -30 | + |
| ZZ- FCR -31 | − |
| ZZ- FCR -32 | + |
| ZZ- FCR -33 | − |
| ZZ- FCR -34 | − |
| ZZ- FCR -35 | − |
| ZZ- FCR -36 | + |
| ZZ- FCR -37 | + |
| ZZ- FCR -38 | + |
| ZZ- FCR -39 | − |
| ZZ- FCR -40 | − |
| ZZ- FCR -41 | − |
| ZZ- FCR -42 | + |
| ZZ- FCR -43 | − |
| ZZ- FCR -44 | − |
| ZZ- FCR -45 | − |
| ZZ- FCR -46 | − |
| ZZ- FCR -47 | − |
| ZZ- FCR -48 | − |
| ZZ- FCR -49 | − |
| ZZ- FCR -50 | − |
| ZZ- FCR -51 | + |
| ZZ- FCR -52 | + |
| ZZ- FCR -53 | − |
| ZZ- FCR -54 | + |
| ZZ- FCR -55 | − |
| ZZ- FCR -56 | − |
| ZZ- FCR -57 | + |
| ZZ- FCR -58 | − |
| ZZ- FCR -59 | − |
| ZZ- FCR -60 | − |
| ZZ- FCR -61 | − |
| ZZ- FCR -62 | − |
| ZZ- FCR -63 | − |
| ZZ- FCR -64 | + |
| ZZ- FCR -65 | + |
| ZZ- FCR -66 | + |
| ZZ- FCR -67 | − |
| ZZ- FCR -68 | − |
| ZZ- FCR -69 | − |
| ZZ- FCR -70 | + |
| ZZ- FCR -71 | − |
| ZZ- FCR -72 | − |
| ZZ- FCR -73 | − |
| ZZ- FCR -74 | − |
| ZZ- FCR -75 | − |
| ZZ- FCR -76 | − |
| ZZ- FCR -77 | − |
| ZZ- FCR -78 | − |
| ZZ- FCR -79 | + |
| ZZ- FCR -80 | − |
| ZZ- FCR -81 | − |
| ZZ- FCR -82 | − |
| ZZ- FCR -83 | − |
| ZZ- FCR -84 | + |
| ZZ- FCR -85 | − |
| ZZ- FCR -86 | − |
| ZZ- FCR -87 | + |
| ZZ- FCR -88 | + |
| ZZ- FCR -89 | − |
| ZZ- FCR -90 | − |
| ZZ- FCR -91 | − |
| ZZ- FCR -92 | − |
| ZZ- FCR -93 | − |
| ZZ- FCR -94 | − |
| ZZ- FCR -95 | − |
| ZZ- FCR -96 | − |
| ZZ- FCR -97 | − |
| ZZ- FCR -98 | − |
| ZZ- FCR -99 | − |
| ZZ- FCR -100 | − |
| ZZ- FCR -101 | + |
| ZZ- FCR -102 | − |
| ZZ- FCR -103 | + |
| ZZ- FCR -104 | − |
| ZZ- FCR -105 | − |
| ZZ- FCR -106 | − |
| ZZ- FCR -107 | − |
| ZZ- FCR -108 | − |
| ZZ- FCR -109 | + |
| ZZ- FCR -110 | + |
| ZZ- FCR -111 | − |
| ZZ- FCR -112 | − |
| ZZ- FCR -113 | − |
| ZZ- FCR -114 | − |
| ZZ- FCR -115 | − |
| ZZ- FCR -116 | − |
| ZZ- FCR -117 | − |
| ZZ- FCR -118 | − |
| ZZ- FCR -119 | − |
| ZZ- FCR -120 | − |
| ZZ- FCR -121 | − |
| ZZ- FCR -122 | + |
| ZZ- FCR -123 | − |
| ZZ- FCR -124 | − |
| ZZ- FCR -125 | − |
| ZZ- FCR -126 | + |
| ZZ- FCR -127 | − |
| ZZ- FCR -128 | − |
| ZZ- FCR -129 | − |
| ZZ- FCR -130 | − |
| ZZ- FCR -131 | + |
| ZZ- FCR -132 | − |
| ZZ- FCR -133 | + |
| ZZ- FCR -134 | + |
| ZZ- FCR -135 | + |
| ZZ- FCR -136 | − |
| ZZ- FCR -137 | − |
| ZZ- FCR -138 | + |
| ZZ- FCR -139 | − |
| ZZ- FCR -140 | − |
| ZZ- FCR -141 | − |
| ZZ- FCR -142 | − |
| ZZ- FCR -143 | − |
| ZZ- FCR -144 | − |
| ZZ- FCR -145 | − |
| ZZ- FCR -146 | − |
| ZZ- FCR -147 | + |
| ZZ- FCR -148 | − |
| ZZ- FCR -149 | − |
| ZZ- FCR -150 | + |
| ZZ- FCR -151 | − |
| ZZ- FCR -152 | + |
| ZZ- FCR -153 | − |
| ZZ- FCR -154 | + |
| ZZ- FCR -155 | − |
| ZZ- FCR -156 | + |
| ZZ- FCR -157 | + |
| ZZ- FCR -158 | + |
| ZZ- FCR -159 | + |
| ZZ- FCR -160 | − |
| ZZ- FCR -161 | − |
| ZZ- FCR -162 | + |
| ZZ- FCR -163 | + |
| ZZ- FCR -164 | + |
| ZZ- FCR -165 | − |
| ZZ- FCR -166 | − |
| ZZ- FCR -167 | − |
| ZZ- FCR -168 | − |
| ZZ- FCR -169 | − |
| ZZ- FCR -170 | − |
| ZZ- FCR -171 | − |
| ZZ- FCR -172 | − |
| ZZ- FCR -173 | − |
| ZZ- FCR -174 | − |
| ZZ- FCR -175 | − |
| ZZ- FCR -176 | − |
| ZZ- FCR -177 | − |
| ZZ- FCR -178 | + |
| ZZ- FCR -179 | + |
| ZZ- FCR -180 | − |
| ZZ- FCR -181 | − |
| ZZ- FCR -182 | + |
| ZZ- FCR -183 | − |
| ZZ- FCR -184 | − |
| ZZ- FCR -185 | + |
| ZZ- FCR -186 | − |
| ZZ- FCR -187 | − |
| ZZ- FCR -188 | − |
| ZZ- FCR -189 | − |
| ZZ- FCR -190 | − |
| ZZ- FCR -191 | − |
| ZZ- FCR -192 | − |
| ZZ- FCR -193 | − |
| ZZ- FCR -194 | − |
| ZZ- FCR -195 | − |
| ZZ- FCR -196 | − |
| ZZ- FCR -197 | − |
| ZZ- FCR -198 | + |
| ZZ- FCR -199 | + |
| ZZ- FCR -200 | − |
| ZZ- FCR -201 | − |
| ZZ- FCR -202 | − |
| ZZ- FCR -203 | − |
| ZZ- FCR -204 | − |
| ZZ- FCR -205 | − |
| ZZ- FCR -206 | − |
| ZZ- FCR -207 | − |
| ZZ- FCR -208 | − |
| ZZ- FCR -209 | − |
| ZZ- FCR -210 | − |
| ZZ- FCR -211 | − |
| ZZ- FCR -212 | − |
| ZZ- FCR -213 | − |
| ZZ- FCR -214 | − |
| ZZ- FCR -215 | − |
| ZZ- FCR -216 | − |
| ZZ- FCR -217 | − |
| ZZ- FCR -218 | − |
| ZZ- FCR -219 | − |
| ZZ- FCR -220 | − |
| ZZ- FCR -221 | − |
| ZZ- FCR -222 | − |
| ZZ- FCR -223 | + |
| ZZ- FCR -224 | + |
| ZZ- FCR -225 | − |
| ZZ- FCR -226 | − |
| ZZ- FCR -227 | − |
| ZZ- FCR -228 | − |
| ZZ- FCR -229 | − |
| ZZ- FCR -230 | − |
| ZZ- FCR -231 | − |
| ZZ- FCR -232 | − |
| ZZ- FCR -233 | − |
| ZZ- FCR -234 | − |
| ZZ- FCR -235 | − |
| ZZ- FCR -236 | − |
| ZZ- FCR -237 | − |
| ZZ- FCR -238 | + |
| ZZ- FCR -239 | − |
| ZZ- FCR -240 | − |
| ZZ- FCR -241 | + |
| ZZ- FCR -242 | − |
| ZZ- FCR -243 | − |
| ZZ- FCR -244 | − |
| ZZ- FCR -245 | + |
| ZZ- FCR -246 | − |
| ZZ- FCR -247 | + |
| ZZ- FCR -248 | + |
| ZZ- FCR -249 | − |
| ZZ- FCR -250 | + |
| ZZ- FCR -251 | − |
| ZZ- FCR -252 | − |
| ZZ- FCR -253 | − |
| ZZ- FCR -254 | − |
| ZZ- FCR -255 | − |
| ZZ- FCR -256 | + |
| ZZ- FCR -257 | − |
| ZZ- FCR -258 | − |
| ZZ- FCR -259 | − |
| ZZ- FCR -260 | − |
| ZZ- FCR -261 | − |
| ZZ- FCR -262 | − |
| ZZ- FCR -263 | − |
| ZZ- FCR -264 | − |
| ZZ- FCR -265 | − |
| ZZ- FCR -266 | − |
| ZZ- FCR -267 | + |
| ZZ- FCR -268 | + |
| ZZ- FCR -269 | + |
| ZZ- FCR -270 | + |
| ZZ- FCR -271 | − |
| ZZ- FCR -272 | − |
| ZZ- FCR -273 | − |
| ZZ- FCR -274 | − |
| ZZ- FCR -275 | + |
| ZZ- FCR -276 | − |
| ZZ- FCR -277 | − |
| ZZ- FCR -278 | − |
| ZZ- FCR -279 | − |
| ZZ- FCR -280 | − |
| ZZ- FCR -281 | − |
| ZZ- FCR -282 | − |
| ZZ- FCR -283 | − |
| ZZ- FCR -284 | − |
| ZZ- FCR -285 | − |
| ZZ- FCR -286 | − |
| ZZ- FCR -287 | − |
| ZZ- FCR -288 | − |
| ZZ- FCR -289 | + |
| ZZ- FCR -290 | − |
| ZZ- FCR -291 | + |
| ZZ- FCR -292 | + |
| ZZ- FCR -293 | + |
| ZZ- FCR -294 | − |
| ZZ- FCR -295 | − |
| ZZ- FCR -296 | − |
| ZZ- FCR -297 | − |
| ZZ- FCR -298 | − |
| ZZ- FCR -299 | − |
| ZZ- FCR -300 | − |
| ZZ- FCR -301 | − |
| ZZ- FCR -302 | − |
| ZZ- FCR -303 | − |
| ZZ- FCR -304 | − |
| ZZ- FCR -305 | − |
| ZZ- FCR -306 | − |
| ZZ- FCR -307 | − |
| ZZ- FCR -308 | − |
| ZZ- FCR -309 | − |
| ZZ- FCR -310 | − |
| ZZ- FCR -311 | + |
| ZZ- FCR -312 | + |
| ZZ- FCR -313 | − |
| ZZ- FCR -314 | + |
| ZZ- FCR -315 | − |
| ZZ- FCR -316 | + |
| ZZ- FCR -317 | − |
| ZZ- FCR -318 | − |
| ZZ- FCR -319 | − |
| ZZ- FCR -320 | − |
| ZZ- FCR -321 | − |
| ZZ- FCR -322 | − |
| ZZ- FCR -323 | − |
| ZZ- FCR -324 | − |
| ZZ- FCR -325 | − |
| ZZ- FCR -326 | − |
| ZZ- FCR -327 | − |
| ZZ- FCR -328 | − |
| ZZ- FCR -329 | − |
| ZZ- FCR -330 | − |
| ZZ- FCR -331 | + |
| ZZ- FCR -332 | + |
| ZZ- FCR -333 | − |
| ZZ- FCR -334 | − |
| ZZ- FCR -335 | + |
| ZZ- FCR -336 | − |
| ZZ- FCR -337 | − |
| ZZ- FCR -338 | + |
| ZZ- FCR -339 | − |
| ZZ- FCR -340 | − |
| ZZ- FCR -341 | − |
| ZZ- FCR -342 | − |
| ZZ- FCR -343 | + |
| ZZ- FCR -344 | − |
| ZZ- FCR -345 | − |
| ZZ- FCR -346 | − |
| ZZ- FCR -347 | − |
| ZZ- FCR -348 | − |
| ZZ- FCR -349 | − |
| ZZ- FCR -350 | − |
| ZZ- FCR -351 | − |
| ZZ- FCR -352 | − |
| ZZ- FCR -353 | + |
| ZZ- FCR -354 | + |
| ZZ- FCR -355 | + |
| ZZ- FCR -356 | − |
| ZZ- FCR -357 | − |
| ZZ- FCR -358 | − |
| ZZ- FCR -359 | + |
| ZZ- FCR -360 | − |
| ZZ- FCR -361 | − |
| ZZ- FCR -362 | − |
| ZZ- FCR -363 | − |
| ZZ- FCR -364 | − |
| ZZ- FCR -365 | − |
| ZZ- FCR -366 | − |
| ZZ- FCR -367 | − |
| ZZ- FCR -368 | − |
| ZZ- FCR -369 | − |
| ZZ- FCR -370 | − |
| ZZ- FCR -371 | − |
| ZZ- FCR -372 | + |
| ZZ- FCR -373 | + |
| ZZ- FCR -374 | − |
| ZZ- FCR -375 | − |
| ZZ- FCR -376 | + |
| ZZ- FCR -377 | − |
| ZZ- FCR -378 | − |
| ZZ- FCR -379 | − |
| ZZ- FCR -380 | − |
| ZZ- FCR -381 | + |
| ZZ- FCR -382 | − |
| ZZ- FCR -383 | − |
| ZZ- FCR -384 | − |
| ZZ- FCR -385 | − |
| ZZ- FCR -386 | − |
| ZZ- FCR -387 | − |
| ZZ- FCR -388 | − |
| ZZ- FCR -389 | − |
| ZZ- FCR -390 | − |
| ZZ- FCR -391 | + |
| ZZ- FCR -392 | + |
| ZZ- FCR -393 | − |
| ZZ- FCR -394 | − |
| ZZ- FCR -395 | − |
| ZZ- FCR -396 | − |
| ZZ- FCR -397 | + |
| ZZ- FCR -398 | − |
| ZZ- FCR -399 | − |
| ZZ- FCR -400 | − |
| ZZ- FCR -401 | + |
| ZZ- FCR -402 | − |
| ZZ- FCR -403 | − |
| ZZ- FCR -404 | − |
| ZZ- FCR -405 | − |
| ZZ- FCR -406 | − |
| ZZ- FCR -407 | − |
| ZZ- FCR -408 | − |
| ZZ- FCR -409 | − |
| ZZ- FCR -410 | − |
| ZZ- FCR -411 | + |
| ZZ- FCR -412 | + |
| ZZ- FCR -413 | − |
| ZZ- FCR -414 | − |
| ZZ- FCR -415 | − |
| ZZ- FCR -416 | + |
| ZZ- FCR -417 | − |
| ZZ- FCR -418 | − |
| ZZ- FCR -419 | − |
| ZZ- FCR -420 | − |
| ZZ- FCR -421 | − |
| ZZ- FCR -422 | − |
| ZZ- FCR -423 | − |
| ZZ- FCR -424 | − |
| ZZ- FCR -425 | − |
| ZZ- FCR -426 | − |
| ZZ- FCR -427 | − |
| ZZ- FCR -428 | − |
| ZZ- FCR -429 | − |
| ZZ- FCR -430 | + |
| ZZ- FCR -431 | + |
| ZZ- FCR -432 | − |
| ZZ- FCR -433 | − |
| ZZ- FCR -434 | − |
| ZZ- FCR -435 | − |
| ZZ- FCR -436 | − |
| ZZ- FCR -437 | − |
| ZZ- FCR -438 | − |
| ZZ- FCR -439 | − |
| ZZ- FCR -440 | − |
| ZZ- FCR -441 | − |
| ZZ- FCR -442 | + |
| ZZ- FCR -443 | − |
| ZZ- FCR -444 | − |
| ZZ- FCR -445 | − |
| ZZ- FCR -446 | − |
| ZZ- FCR -447 | + |
| ZZ- FCR -448 | + |
| ZZ- FCR -449 | − |
| ZZ- FCR -450 | − |
| ZZ- FCR -451 | − |
| ZZ- FCR -452 | − |
| ZZ- FCR -453 | − |
| ZZ- FCR -454 | − |
| ZZ- FCR -455 | − |
| ZZ- FCR -456 | − |
| ZZ- FCR -457 | − |
| ZZ- FCR -458 | − |
| ZZ- FCR -459 | − |
| ZZ- FCR -460 | − |
| ZZ- FCR -461 | − |
| ZZ- FCR -462 | − |
| ZZ- FCR -463 | − |
| ZZ- FCR -464 | − |
| ZZ- FCR -465 | − |
| ZZ- FCR -466 | − |
| ZZ- FCR -467 | − |
| ZZ- FCR -468 | − |
| ZZ- FCR -469 | − |
| ZZ- FCR -470 | + |
| ZZ- FCR -471 | − |
| ZZ- FCR -472 | + |
| ZZ- FCR -473 | + |
| ZZ- FCR -474 | − |
| ZZ- FCR -475 | − |
| ZZ- FCR -476 | + |
| ZZ- FCR -477 | − |
| ZZ- FCR -478 | − |
| ZZ- FCR -479 | − |
| ZZ- FCR -480 | − |
| ZZ- FCR -481 | − |
| ZZ- FCR -482 | − |
| ZZ- FCR -483 | − |
| ZZ- FCR -484 | − |
| ZZ- FCR -485 | − |
| ZZ- FCR -486 | + |
| ZZ- FCR -487 | + |
| ZZ- FCR -488 | − |
| ZZ- FCR -489 | − |
| ZZ- FCR -490 | − |
| ZZ- FCR -491 | − |
| ZZ- FCR -492 | − |
| ZZ- FCR -493 | − |
| ZZ- FCR -494 | + |
| ZZ- FCR -495 | − |
| ZZ- FCR -496 | − |
| ZZ- FCR -497 | − |
| ZZ- FCR -498 | − |
| ZZ- FCR -499 | − |
| ZZ- FCR -500 | − |

Note:
+ represents presence of the FCR-1B-d8 molecular marker; and − represents absence of the FCR-1B-d8 molecular marker.

The embodiments described above are merely descriptions of implementation methods of the disclosure, and are not intended to limit a scope of the disclosure. Without departing from a design spirit of the disclosure, various modifications and improvements made by those skilled in the art to the technical solutions of the disclosure should fall within a scope of protection defined in claims of the disclosure.

What is claimed is:

1. A derived cleaved amplified polymorphic sequence (dCAPS) molecular marker for identifying resistance of wheat to Fusarium crown rot caused by Fusarium pseudograminearum, wherein the dCAPS molecular marker is located at a site of 676077050 to 676084414 base pairs (bp) on a wheat chromosome 1B.

2. A primer set, wherein the primer set is configured to amplify the dCAPS molecular marker as claimed in claim 1.

3. The primer set as claimed in claim 2, wherein the primer set comprises: an upstream primer and a downstream primer; and the nucleotide sequence of the upstream primer is shown as SEQ ID NO: 1, and the nucleotide sequence of the downstream primer is shown as SEQ ID NO: 2.

4. A detection reagent for identifying the dCAPS molecular marker, wherein the detection reagent comprises the primer set as claimed in claim 2.

5. A detection kit for identifying the dCAPS molecular marker, wherein the detection kit comprises the primer set as claimed in claim 2.

6. A detection kit for identifying the dCAPS molecular marker, wherein the detection kit comprises the detection reagent as claimed in claim 4.

7. An application method of the dCAPS molecular marker as claimed in claim 1, comprising:
identifying the resistance of wheat to the Fusarium crown rot caused by the Fusarium pseudograminearum by using the dCAPS molecular marker.

8. A method for identifying the resistance of wheat to the Fusarium crown rot caused by the Fusarium pseudograminearum, comprising:
taking a genomic DNA of a wheat sample as a template, performing a polymerase chain reaction (PCR) amplification with the primer set as claimed in claim 2 to obtain a PCR amplification product, digesting the PCR amplification product to obtain a digested product, and performing an electrophoresis on the digested product to obtain an electrophoresis product; when the electrophoresis product comprises a DNA fragment with 169 bp, indicating that the wheat sample is a resistant variety to the Fusarium crown rot caused by the Fusarium pseudograminearum;
wherein steps of the digesting specifically comprise:
mixing 0.5 microliters (μL) of 10 units per microliter (U/μL) of PvuII enzyme, 1.0 μL of 10×M buffer, 1.0 μL of the PCR amplification product and 7.5 μL of double-distilled water (ddH₂O) to obtain a mixture, digesting the mixture at a temperature of 37.0 Celsius degree (C.) for 4 hours (h) to obtain a digested mixture, and adding 1.0 μL of loading buffer into the digested mixture to obtain the digested product; and
wherein steps of the electrophoresis comprise:
performing a polyacrylamide gel electrophoresis with a mass fraction of 8% on 2 μL of the digested product to obtain the electrophoresis product;
when the electrophoresis product comprises the DNA fragment with 169 bp, indicating that the wheat sample has a target dCAPS molecular marker, and predicting that the wheat sample has resistance to the Fusarium crown rot;
when the electrophoresis product does not comprise the DNA fragment with 169 bp, indicating that the wheat sample does not have the resistance to the Fusarium crown rot.


SEQUENCE LISTING (2 sequences)

SEQ ID NO: 1 - DNA, 25 bp, synthetic construct
  source 1..25: mol_type = other DNA
tttgcttagtttattaaaagcagct

SEQ ID NO: 2 - DNA, 20 bp, synthetic construct
  source 1..20: mol_type = other DNA
cactctgttcccatcatcac